(19) United States
(12) Patent Application Publication
(10) Pub. No.: US 2020/0405823 A1
(43) Pub. Date: Dec. 31, 2020

(54) A HEPATOCYTE-MIMICKING ANTIDOTE FOR ALCOHOL INTOXICATION

(71) Applicants: The Regents of the University of California, Oakland, CA (US); THE UNIVERSITY OF SOUTHERN CALIFORNIA, Los Angeles, CA (US)

(72) Inventors: Yunfeng Lu, Culver City, CA (US); Cheng Ji, Los Angeles, CA (US); Duo Xu, Los Angeles, CA (US); Hui Han, Los Angeles, CA (US)

(73) Assignee: The Regents of the University of California, Oakland, CA (US)

(21) Appl. No.: 16/980,582

(22) PCT Filed: Mar. 29, 2019
(86) PCT No.: PCT/US19/24983

§ 371 Date: Sep. 14, 2020

Related U.S. Application Data
(60) Provisional application No. 62/650,040, filed on Mar. 29, 2018.

(51) Int. Cl. A61K 38/44; A61K 31/7084; A61K 9/00; A61K 9/50

(52) U.S. Cl. CPC A61K 38/443; A61K 38/44; A61K 9/5021; A61K 9/0053; A61K 9/0019; A61K 31/7084

(57) ABSTRACT

Alcohol intoxication causes serious diseases, whereas current treatments are mostly supportive and unable to remove alcohol efficiently. Upon alcohol consumption, alcohol is sequentially oxidized to acetaldehyde and acetate by the endogenous alcohol dehydrogenase and aldehyde dehydrogenase, respectively. We disclose a hepatocyte-mimicking antidote for alcohol intoxication through the co-delivery of the nanocapsules of alcohol oxidase (AOx), catalase (CAT), and aldehyde dehydrogenase (ALDH) to the liver, where AOx and CAT catalyze the oxidation of alcohol to acetaldehyde, while ALDH catalyzes the oxidation of acetaldehyde to acetate. Administered to alcohol-intoxicated mice, the antidote rapidly accumulates in the liver and enables a significant reduction of the blood alcohol concentration. Moreover, blood acetaldehyde concentration is maintained at an extremely low level, significantly contributing to liver protection. Such an antidote, which can eliminate alcohol and acetaldehyde simultaneously, holds great promise for the treatment of alcohol intoxication and poisoning.

### CROSS-REFERENCE TO RELATED APPLICATIONS

This application claims priority under Section 119(e) from U.S. Provisional Application Ser. No. 62/650,040 filed Mar. 29, 2018, entitled “A HEPATOCYTE-MIMICKING ANTIDOTE FOR ALCOHOL INTOXICATION” the contents of which are incorporated herein by reference.

### STATEMENT AS TO RIGHTS TO INVENTIONS MADE UNDER FEDERALLY SPONSORED RESEARCH AND DEVELOPMENT

This invention was made with government support under Grant Number AA023952, awarded by the National Institutes of Health. The Government has certain rights in the invention.

### TECHNICAL FIELD

This disclosure relates to encapsulated enzyme nanocomplexes designed to metabolize alcohol and alcohol metabolites.

### BACKGROUND OF THE INVENTION

Alcohol consumption is a millennium-old fashion of human civilization, while excessive use of alcohol causes serious diseases and health problems, such as injury, gastrointestinal and hepatic diseases, cancer, and cardiovascular disease. Among people aged 15-49 years, alcohol consumption is the leading risk factor for premature mortality and disability. Although acute alcohol intoxication takes up 8-10% of emergency room administrations, current treatments (e.g., homeostasis management and prevention of complications) are mostly supportive and still rely on the endogenous enzymes to eliminate alcohol. To date, there are no effective antidotes for alcohol intoxication yet.

Despite the development of colloidal antidotes, small molecule drugs, and inorganic nanoparticles for alcohol detoxification, their inability to actively eliminate alcohol limits their therapeutic efficacy. In view of the variety of problems associated with alcohol consumption, intoxication and abuse, there is a need for methods and materials that can reduce the concentrations of ethanol in vivo. Such methods and materials are useful, for example, in treating or ameliorating pathological conditions associated with the consumption of alcohol, including acute alcohol intoxication as well as treating alcohol abuse and dependence.

### SUMMARY OF THE INVENTION

Inspired by the metabolism of alcohol, we show that the effective removal of alcohol and acetaldehyde in vivo can be achieved by the co-delivery of alcohol oxidase (AOx), catalase (CAT), and aldehyde dehydrogenase (ALDH) to the liver. In this invention, AOx and CAT in the form of an enzyme complex, as well as ALDH, are encapsulated within a cationic polymer shell through in situ polymerization, which forms enzyme nanocapsules denoted as n(AOx-CAT) and n(ALDH), respectively. The polymer shells stabilize the enzymes while allowing the fast transport of the substrates, rendering the enzyme nanocapsules with highly retained activity and enhanced stability. Similar to other positively-charged nanoparticles, the nanocapsules disclosed herein can be effectively delivered to the liver through intravenous administration, where n(AOx-CAT) converts alcohol to acetaldehyde and hydrogen peroxide (H₂O₂), with the latter removed by the CAT. Acetaldehyde generated in these reactions is then converted to acetate by n(ALDH), for example in the presence of NAD⁺.

The invention disclosed herein has a number of embodiments. One embodiment of the invention is a method of decreasing the concentration of ethanol and its metabolites in an individual. Typically, this method comprises the steps of administering a multiple-enzyme nanocomplex system to the individual, wherein the multiple-enzyme nanocomplex system comprises an alcohol oxidase enzyme that generates hydrogen peroxide and acetaldehyde a first enzymatic reaction with ethanol and a catalase enzyme that converts the hydrogen peroxide into water in a second enzymatic reaction. In this method, the alcohol oxidase and the catalase are disposed within a polymeric network configured to form a shell that encapsulates the alcohol oxidase and the catalase. In this method, aldehyde dehydrogenase enzyme is also administered to the individual (e.g. aldehyde dehydrogenase disposed within a polymeric network configured to form a shell that encapsulates the aldehyde dehydrogenase) in order to converts acetaldehyde to acetate in a third enzymatic reaction. In this methodology, the alcohol oxidase, catalase and aldehyde dehydrogenase are disposed in an environment that allow them to react with ethanol and its metabolites in the individual, so that the concentration of ethanol and its metabolites in the individual is decreased.

Certain embodiments of this methodology for decreasing the concentration of ethanol in an individual further comprise administering nicotinamide adenine dinucleotide (NAD). Optionally, the nicotinamide adenine dinucleotide is disposed within a polymeric network configured to form a shell that encapsulates the nicotinamide adenine dinucleotide. In some embodiments of the invention, the alcohol oxidase enzyme, the catalase enzyme and/or the aldehyde dehydrogenase enzyme is coupled to a polymeric shell or an enzyme within a polymeric shell. Typically in these embodiments, the polymeric network encapsulates the alcohol oxidase and/or the catalase and/or the aldehyde dehydrogenase and or the nicotinamide adenine dinucleotide in a manner that inhibits their degradation when disposed in an in vivo environment.

Another embodiment of the invention is a composition of matter comprising a multiple-enzyme nanocomplex for use in a patient for the treatment of a condition resulting from the consumption of alcohol. In such compositions, a multiple-enzyme nanocomplex can comprise an alcohol oxidase enzyme that generates hydrogen peroxide and acetaldehyde in a first enzymatic reaction with alcohol and a catalase enzyme that converts the hydrogen peroxide into water in a second enzymatic reaction. Such compositions can also comprise an aldehyde dehydrogenase enzyme that converts acetaldehyde to acetate in a third enzymatic reaction. Typically in these embodiments, one or more of these enzymes is disposed within a polymeric network configured to form a shell that encapsulates the enzymes. The polymeric network encapsulating the one or more enzymes is formed to exhibit a permeability sufficient to allow the alcohol to diffuse from an external environment outside of the shell to the alcohol oxidase. In certain embodiments of the invention, the alcohol oxidase, the catalase and/or the aldehyde dehydrogenase is coupled to a polymeric shell or another enzyme within a polymeric shell. Optionally the composition further comprises nicotinamide adenine dinucleotide (e.g. nicotinamide adenine dinucleotide disposed within a polymeric network configured to form a shell that encapsulates the nicotinamide adenine dinucleotide).

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIGS. 1A and 1B provide schematics showing the design of a hepatocyte-mimicking antidote for alcohol intoxication. FIG. 1(a) provides a schematic showing alcohol metabolism in hepatocytes. Cytosolic ADH converts alcohol to acetaldehyde with the cofactor NAD⁺ (Step 1). Then, ALDH in the mitochondria converts acetaldehyde to acetate with NAD⁺ (Step 2). FIG. 1(b) provides a schematic of the synthesis of n(AOx-CAT) and n(ALDH) through in situ polymerization. • and • • represent monomers and crosslinkers. Then, n(AOx-CAT) and n(ALDH) are co-delivered to the liver cells, where they catalyze the consecutive oxidation of alcohol to acetaldehyde, then to acetate.

FIGS. 2A-2F provide photographs and graphed data illustrating characterizations of the nanocapsules. FIG. 2(a): Transmission electron microscopy images of n(AOx-CAT) and n(ALDH) with uniform diameters of 32.8±4.0 nm and 34.3±3.9 nm, respectively. FIG. 2(b) Size and FIG. 2(c) Zeta potentials of n(AOx-CAT) and n(ALDH) measured by dynamic light scattering. FIG. 2(d) The kinetics of the removal of alcohol and acetaldehyde in a closed system containing alcohol (0.4%, w/v), after incubating with PBS, or n(AOx-CAT) (0.8 U/mL), or n(ALDH) (6.0 U/mL), or the mixture of n(AOx-CAT) and n(ALDH) for 4 hr. FIG. 2(e) Reduced cytotoxicity in primary mouse hepatocytes (PMH) after the simultaneous removal of alcohol and acetaldehyde. Cytotoxicity was assessed by measuring the release of lactate dehydrogenase. FIG. 2(f) Reduced apoptosis in PMH after the simultaneous removal of alcohol and acetaldehyde. Apoptosis was indicated by the relative luminescent unit (RLU) of Caspase 3/7 activity. Data are presented as mean SEM (=3˜6). **P<0.01, ***P<0.005 and ****P<0.0001.

FIGS. 3A-3D provide photographs and graphed data illustrating the delivery and therapeutic efficacy of n(AOx-CAT) and n(ALDH) as the antidote. FIG. 3(a) Confocal laser scanning microscopy (CLSM) images of mouse hepatocytes (AML12) after 4 hr incubation with the native AOx-CAT and ALDH, or n(AOx-CAT) and n(ALDH). Hoechst 33342 was used to stain the nuclei. The native AOx-CAT and n(AOx-CAT) were labeled with TAMRA; the native ALDH and n(ALDH) were labeled with FL Scale bar, 50 μm. FIG. 3(b) Fluorescence imaging of the major organs after intravenous administration of n(AOx-CAT) and n(ALDH). For imaging purpose, n(AOx-CAT) and n(ALDH) were labeled with TAMRA and AF680, respectively. FIG. 3(c), FIG. 3(d) Blood alcohol concentrations (BAC) FIG. 3(c) and blood acetaldehyde concentrations (BAchC) (d) of alcohol-intoxicated mice treated with PBS, n(AOx-CAT) and n(ALDH), or n(AOx-CAT) and n(ALDH) with NAD⁺. Mice were gavaged with alcohol at 5 mg/g body weight, and BAC were measured at 30, 120, 240, and 420 min. Data are presented as mean±SEM (n=6˜9). *P<0.05, **P<0.01, and ****P<0.0001.

FIGS. 4A-4E provide photographs and graphed data illustrating the biocompatibility of the antidote after HFD and acute alcohol intoxication. FIG. 4(a) Representative H&E and Oil Red O staining of the liver tissues in alcohol-intoxicated mice treated with PBS, or n(AOx-CAT) and n(ALDH) with NAD⁺ as the antidote. Liver tissue from healthy mice was used as the control. Scale bar, 50 μm. FIG. 4(b) Total liver triglycerides in healthy mice (n=5) and alcohol-intoxicated mice treated with PBS (n=5) or the antidote (n=7). FIG. 4(c) Plasma ALT level in healthy mice (n=5) and alcohol-intoxicated mice treated with PBS (n=5) or the antidote (n=7). FIG. 4(d) Protein expression levels of the ER stress markers (GRP78, CHOP), and autophagy markers including the mechanistic target of rapamycin (mTOR), phosphorylated mTOR (pmTOR) and microtubule-associated protein 1A/1B-light chain 3 (LC3B). FIG. 4(e) Quantification of protein expression levels of the ER stress and autophagy markers, normalized with glyceraldehyde-3-phosphate dehydrogenase (GAPDH). Data are presented as mean SEM (n=5˜7).

FIGS. 5A-5I provide photographs and graphed data illustrating aspects of the invention. FIG. 5(a) The reaction used for the determination of acetaldehyde concentration. FIG. 5(b) UV/Vis spectra of MBTH-acetaldehyde adducts at different concentrations. FIG. 5(c) The standard curve based on the absorption at 600 nm. FIG. 5(d), FIG. 5(e) Thermal stability of the native AOx-CAT and n(AOx-CAT) (d), and the native ALDH and n(ALDH) FIG. 5(e). FIG. 5(f), FIG. 5(g) Proteolytic stability of the native AOx-CAT and n(AOx-CAT) (f), and the native ALDH and n(ALDH) FIG. 5(g). FIG. 5(h) Long-term stability of n(AOx-CAT) and n(ALDH) in PBS (pH 7.4) at 4° C. within 2 weeks. FIG. 5(i) The polydispersity index of n(AOx-CAT) and n(ALDH) within the 2-week stability measurement.

FIG. 6 provides graphed data showing fluorescence spectrum of n(AOx-CAT) and the mixture of AOx and CAT. AOx and CAT were labeled with fluorescein (FL) and tetramethylrhodamine (TAMRA), respectively. The excitation wavelength was 450 nm.

FIG. 7 provides graphed data showing the production of hydrogen peroxide (H₂O₂) measured by HRP/TMB assay.

FIGS. 8A-8B provide graphed data showing aspects of the invention. FIG. 8(a) HeLa cell viability after incubating with n(AOx-CAT), or n(ALDH), or the mixture of n(AOx-CAT) and n(ALDH) at different concentrations for 24 hr. FIG. 8(b) Decrease in the endoplasmic reticulum (ER) stress response after the removal of acetaldehyde by n(ALDH), as evaluated by the mRNA expression of ER stress markers: glucose-regulated protein 78 (GRP78), C/EBP homologous protein (CHOP) and alternatively spliced X-box binding protein 1 (sXBP1).

FIGS. 9A-9B provide photographs showing aspects of the invention. Hepatocyte (AML12) uptake of the native enzymes or nanocapsules. FIG. 9(a) CLSM images of AML12 cells incubated with the native AOx-CAT or n(AOx-CAT). FIG. 9(b) CLSM images of AML12 cells incubated with the native ALDH or n(ALDH). The native AOx-CAT and n(AOx-CAT) were labeled with tetramethylrhodamine (TAMRA). The native ALDH and n(ALDH) were labeled with fluorescein (FL). Scale bar, 50 μm.

FIGS. 10A-10B provide photographs showing hepatocyte internalization of the nanocapsules. FIG. 10(a) Z-stacking and FIG. 10(b) z-slicing images of AML12 cells incubated with n(AOx-CAT) and n(ALDH). Scale bar, 50 μm.

FIGS. 11A-11C provide photographs showing macrophage (J774A.1) uptake of the native enzymes or nanocapsules. FIG. 11(a) Fluorescence images of J774A.1 cells incubated with the native AOx-CAT and ALDH, or n(AOx-CAT) and n(ALDH). FIG. 11(b) Fluorescence images of J774A.1 cells incubated with the native AOx-CAT or n(AOx-CAT). FIG. 11(c) Fluorescence images of J774A.1 cells incubated with the native ALDH or n(ALDH). The native AOx-CAT and n(AOx-CAT) were labeled with tetramethylrhodamine (TAMRA). The native ALDH and n(ALDH) were labeled with fluorescein (FL). Scale bar, 50 μm.

FIGS. 12A-12B provide photographs showing the trafficking of nanocapsules through endocytosis. FIG. 12(a) Early endosomes and FIG. 12(b) late endosomes were stained with anti-EEA1 antibody and anti-Rab7 antibody, respectively. J774A.1 cells were incubated with n(ALDH) at 37° C. for 15, 30, 60, and 120 min before imaging with CLSM. Scale bar, 20 μm.

FIGS. 13A-13B provide photographs and graphed data showing aspects of the invention. FIG. 13(a) Biodistribution of nanocapsules in mice measured by fluorescence imaging. n(ALDH) was used as an example of single nanocapsules. FIG. 13(b) Quantification of the fluorescence intensity in each organ at 4 hr and 8 hr.

FIGS. 14A-14C provide photographs and graphed data showing aspects of the invention. FIG. 14(a) Biodistribution of nanocapsules in the major organs of mice, measured by fluorescence imaging. n(AOx-CAT) was used as an example, and 50 μg were administered. FIG. 14(b) Biodistribution of nanocapsules in the major organs of mice, measured by fluorescence imaging. n(AOx-CAT) was used as an example, and 100 μg were administered. FIG. 14(c) ALT levels in mice treated with PBS, 50 μg n(AOx-CAT), and 100 μg n(AOx-CAT). Data are presented as mean SEM (n=3).

FIGS. 15A-15B provide graphed data showing aspects of the invention. FIG. 15(a) Time to LORR. FIG. 15(b) Restoration of consciousness (time of sleep) of alcohol-intoxicated mice with or without the antidote.

FIGS. 16A-16B provide photographs and graphed data showing aspects of the invention. FIG. 16(a) H&E and Oil Red O staining of liver tissues from the alcohol-intoxicated mice treated with n(AOx-CAT) only. Scale bar, 50 μm. FIG. 16(b) Total liver triglyceride content from the alcohol-intoxicated mice treated with n(AOx-CAT) only.

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized and structural changes may be made without departing from the scope of the present invention. Many of the techniques and procedures described or referenced herein are well understood and commonly employed by those skilled in the art. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

The invention provides a hepatocyte-mimicking antidote for alcohol intoxication by the co-delivery of n(AOx-CAT) and n(ALDH) to the liver. While n(AOx-CAT) enables rapid alcohol removal, acetaldehyde generated by AOx-CAT can be efficiently removed by n(ALDH). Administration of the antidote to alcohol-intoxicated mice results in significant reduction in blood alcohol content (BAC) without the accumulation of acetaldehyde. Such an antidote could provide profound therapeutic benefits to alcohol-intoxicated patients, and rescue lives in emergency rooms.

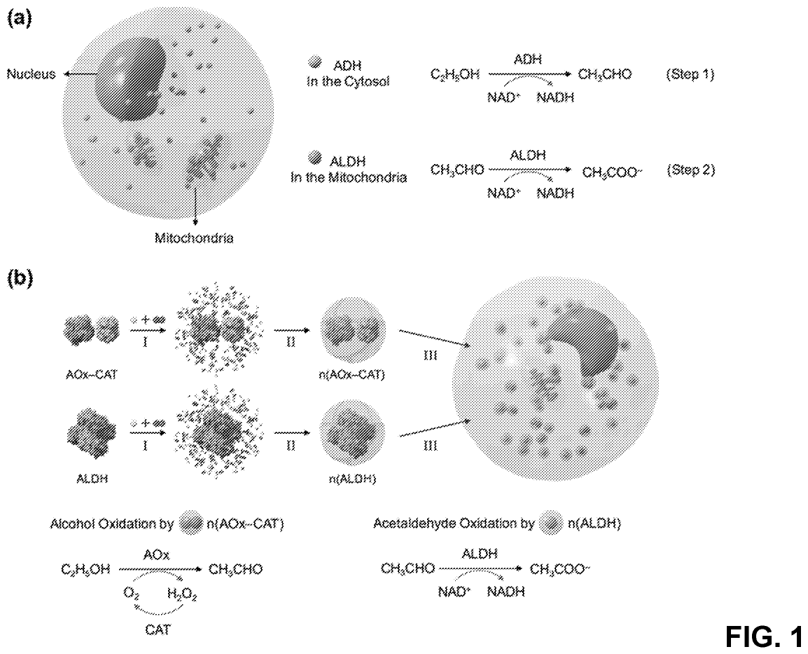

The metabolism of alcohol mainly relies on cytosolic alcohol dehydrogenase (ADH) and mitochondrial aldehyde dehydrogenase (ALDH) in the hepatocytes^{[17,18]}. Cytochrome P450 2E1 in the microsomes only becomes active after a significant amount of alcohol is consumed. ADH and ALDH convert alcohol to acetaldehyde and then to acetate with the help of nicotinamide adenine dinucleotide (NAD) (FIG. 1a). We show that the effective removal of alcohol and acetaldehyde could be achieved by the co-delivery of alcohol oxidase (AOx), catalase (CAT), and ALDH to the liver. As illustrated in FIG. 1b, AOx and CAT in the form of an enzyme complex, as well as ALDH, are encapsulated within a cationic polymer shell through in situ polymerization^{[19,20]}, which forms enzyme nanocapsules denoted as n(AOx-CAT) and n(ALDH), respectively. The polymer shells stabilize the enzymes while allowing fast transport of the substrates, rendering the enzyme nanocapsules with highly retained activity and enhanced stability^{[21,22]}. Similar to other positively-charged nanoparticles, such nanocapsules can be effectively delivered to the liver through intravenous administration^{[23-25]}, where n(AOx-CAT) converts alcohol to acetaldehyde and hydrogen peroxide (11202), with the latter removed by the CAT. As-generated acetaldehyde is then converted to acetate by n(ALDH) with the help of NAD⁺.

ADH and ALDH have been encapsulated within erythrocytes by electroporation^{[26-28]}. Such-enzyme loaded erythrocytes were intravenously administered to alcohol-intoxicated mice, exhibiting a circulation half-life of 4.5 days and leading to a significant decrease in the blood alcohol concentration (BAC)^{[28]}. However, due to the low loading efficiency, it requires the administration of a large number of enzyme-loaded erythrocytes in order to achieve a reasonable reduction in BAC. For instance, given an enzyme loading efficiency of 2.1×10⁻⁹ U ADH or 5.4×10⁻¹¹ U ALDH per erythrocyte^{[28]}, it would take ˜4.8×10⁸ or 1.9×10¹⁰ enzyme-loaded erythrocytes to deliver 1 U of ADH or ALDH. This quantity approximates to the number of erythrocytes in 100 or 4000 mL blood of human. In addition, the short shelf-life of erythrocytes (up to 42 days)^{[29,30]} and the biosafety concerns^{[31]} over the blood specimens further preclude its use for therapeutic purposes.

Our antidote strategy mimics the function of hepatocytes by co-delivering n(AOx-CAT) and n(ALDH) to the liver, where these enzymes are located in close proximity within the cells, enabling the simultaneous and effective breakdown of alcohol and the toxic intermediates (H₂O₂ and acetaldehyde). Furthermore, alcohol oxidation by ADH and ALDH in the liver consumes a substantial amount of NAD⁺, which may result in NAD⁺ deficiency that hinders continuous elimination of alcohol and acetaldehyde. Despite the regeneration of NAD⁺ through mitochondrial respiration, the insufficient availability of NAD⁺ remains as the rate-limiting step in alcohol metabolism^{[32]}. In our biomimetic strategy, in contrast, the majority of NAD⁺ could be used by n(ALDH) for efficient acetaldehyde oxidation, given that n(AOx-CAT) does not require this cofactor. The invention disclosed herein has a number of embodiments. Embodiments of the invention include, for example, methods of decreasing the concentration of ethanol and its metabolites in an individual (e.g. an individual suffering from ethanol intoxication). Such methods typically comprise the steps of administering a multiple-enzyme nanocomplex system to the individual, wherein the multiple-enzyme nanocomplex system comprises an alcohol oxidase enzyme that generates hydrogen peroxide and acetaldehyde a first enzymatic reaction with ethanol and also a catalase enzyme that converts the hydrogen peroxide into water in a second enzymatic reaction; and a polymeric network configured to form a shell that encapsulates the alcohol oxidase and the catalase. Typically in such embodiments, the polymeric network exhibits a permeability sufficient to allow the ethanol to diffuse from an external environment outside of the shell to the alcohol oxidase so that the hydrogen peroxide is generated. In these methods, an aldehyde dehydrogenase enzyme that converts acetaldehyde to acetate in a third enzymatic reaction is also administered in a manner that allows the alcohol oxidase, catalase and aldehyde dehydrogenase to react with ethanol and its metabolites in the individual; so that the concentration of ethanol and its metabolites in the individual is decreased. Optionally the methods, further comprise administering nicotinamide adenine dinucleotide (NAD). In certain embodiments of the invention, the multiple-enzyme nanocomplex system is administered parenterally.

In certain embodiments, the aldehyde dehydrogenase and/or the nicotinamide adenine dinucleotide is disposed within a polymeric network configured to form a shell that encapsulates the aldehyde dehydrogenase and/or the nicotinamide adenine dinucleotide. Optionally, the alcohol oxidase enzyme, the catalase enzyme and/or the aldehyde dehydrogenase enzyme is coupled to a polymeric shell or an enzyme within a polymeric shell. Typically, the multiple-enzyme nanocomplex system reduces blood ethanol concentrations in the individual by at least 25, 50, 75 or 100 mg/dL within 90 minutes following administration to the individual.

Embodiments of the invention also comprise compositions of matter. Typically these compositions comprise a multiple-enzyme nanocomplex system for use in a patient for the treatment of a condition resulting from the consumption of alcohol, wherein the multiple-enzyme nanocomplex system comprises: an alcohol oxidase enzyme that generates hydrogen peroxide and acetaldehyde in a first enzymatic reaction with alcohol; a catalase enzyme that converts the hydrogen peroxide into water in a second enzymatic reaction; an aldehyde dehydrogenase enzyme that converts acetaldehyde to acetate in a third enzymatic reaction; and a polymeric network configured to form a shell that encapsulates the alcohol oxidase and the catalase wherein the polymeric network exhibits a permeability sufficient to allow the alcohol to diffuse from an external environment outside of the shell to the alcohol oxidase. Typically in these compositions, the aldehyde dehydrogenase enzyme is disposed within a polymeric network configured to form a shell that encapsulates only the aldehyde dehydrogenase. Optionally, the alcohol oxidase, the catalase and/or the aldehyde dehydrogenase is coupled to a polymeric shell or another enzyme disposed within a polymeric shell. Certain embodiments of the invention further comprise nicotinamide adenine dinucleotide. Optionally the nicotinamide adenine dinucleotide is disposed within a polymeric network configured to form a shell that encapsulates the nicotinamide adenine dinucleotide. In certain embodiments of the invention, the alcohol oxidase enzyme and catalase enzyme are disposed within the polymeric network at a distance from each other of less than 50, 40, 30, 20 or 10 nm.

Yet another embodiment of the invention is a method of making a pharmaceutical composition comprising combining together in an aqueous formulation a multiple-enzyme nanocomplex system and a pharmaceutical excipient selected from the group consisting of a preservative, a tonicity adjusting agent, a detergent, a viscosity adjusting agent, a sugar or a pH adjusting agent. Typically in these methods, the enzyme nanocomplex system comprises an alcohol oxidase enzyme that generates hydrogen peroxide and acetaldehyde in a first enzymatic reaction with alcohol; a catalase enzyme that converts the hydrogen peroxide into water in a second enzymatic reaction; and an aldehyde dehydrogenase enzyme that converts acetaldehyde to acetate in a third enzymatic reaction. Typically in these methods, a polymeric network is disposed around the alcohol oxidase enzyme and the catalase enzyme and configured to form a shell that encapsulates the alcohol oxidase enzyme and the catalase enzyme; and another polymeric network is disposed around the aldehyde dehydrogenase enzyme and the catalase enzyme and configured to form a shell that encapsulates the aldehyde dehydrogenase enzyme. In some embodiments of the invention, the multiple-enzyme nanocomplex system further comprises nicotinamide adenine dinucleotide (NAD). In certain embodiments of the invention, polymeric shell (e.g. the one encapsulating the aldehyde dehydrogenase enzyme) is formed to comprise moieties capable forming disulfide bonds (e.g. those formed by cysteine residues disposed in crosslinkers that can couple polymer chains together), and said moieties are reduced. In certain embodiments of the invention, the zeta potentials of the polymeric shells are selected to be at least ˜1, ˜2 or ˜4 mV at physiological pH. Optionally in these methods, the pharmaceutical excipient is selected for use in intravenous administration.

For pharmaceutical compositions suitable for administration to humans, the term “excipient” is meant to include, but is not limited to, those ingredients described in Remington: The Science and Practice of Pharmacy, Lippincott Williams & Wilkins, 21st ed. (2006) the contents of which are incorporated by reference herein. The pharmaceutical compositions may also be administered in a variety of ways, for example intravenously. Solutions of the compounds can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the compounds which are adapted for the extemporaneous preparation of sterile injectable or infusible solutions or dispersions. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof.

Useful liquid carriers include water, alcohols or glycols or water/alcohol/glycol blends, in which the compounds can be dissolved or dispersed at effective levels, optionally with the aid of non-toxic surfactants. Adjuvants such as additional antimicrobial agents can be added to optimize the properties for a given use.

Effective dosages and routes of administration of agents of the invention are conventional. The exact amount (effective dose) of the agent will vary from subject to subject, depending on, for example, the species, age, weight and general or clinical condition of the subject, the severity or mechanism of any disorder being treated, the particular agent or vehicle used, the method and scheduling of administration, and the like. A therapeutically effective dose can be determined empirically, by conventional procedures known to those of skill in the art. See e.g., The Pharmacological Basis of Therapeutics, Goodman and Gilman, eds., Macmillan Publishing Co., New York. For example, an, effective dose can be estimated initially either in cell culture assays or in suitable animal models. The animal model may also be used to determine the appropriate concentration ranges and routes of administration. Such information can then be used to determine useful doses and routes for administration in humans. A therapeutic dose can also be selected by analogy to dosages for comparable therapeutic agents.

The particular mode of administration and the dosage regimen will be selected by the attending clinician, taking into account the particulars of the case (e.g., the subject, the disease, the disease state involved, and whether the treatment is prophylactic).

### Aspects and Embodiments of the Invention

Synthesis and Characterization of the Enzyme Nanocapsules.

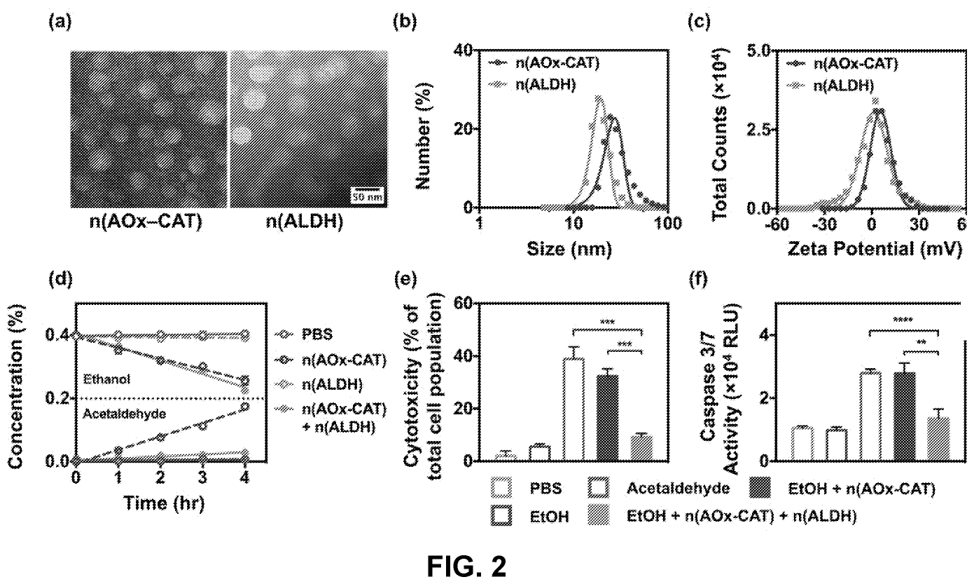

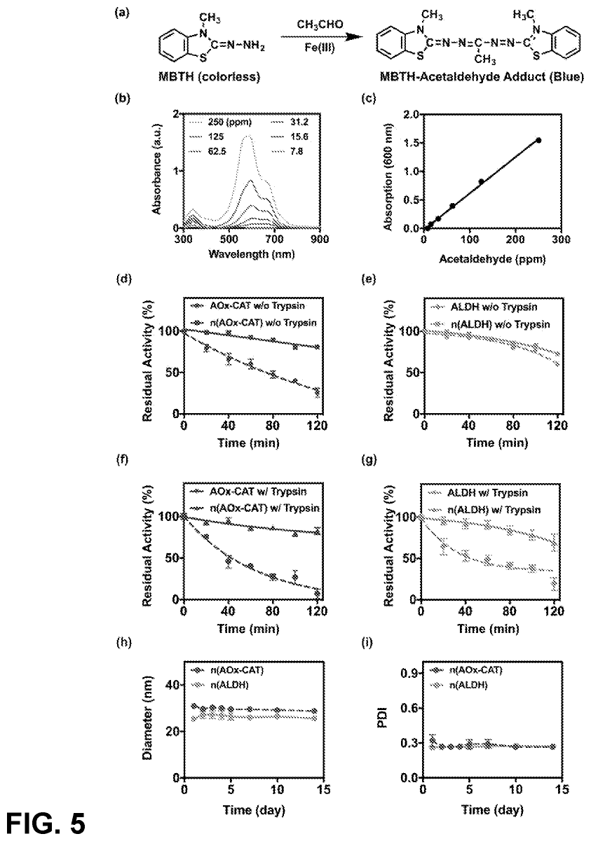

Spherical and monodispersed n(AOx-CAT) and n(ALDH) averaging 32.8±4.0 nm and 34.3±3.9 nm were observed with transmission electron microscopy and dynamic light scattering (FIG. 2a, b). Meanwhile, n(AOx-CAT) and n(ALDH) showed zeta potentials of ˜4 mV and ˜2 mV, respectively (FIG. 2c). The positive zeta potentials would allow their rapid accumulation in the liver after administration^{[23,24,33,34]}. While the native enzymes are found to be unstable under physiological temperature or in the presence of proteases, the polymer shells also enhance the thermal and proteolytic stability of the enzymes. For instance, when incubated at 37° C. for 2 hr, especially in the presence of protease, the native enzymes quickly lost their activity (FIG. 5). On the contrary, both n(AOx-CAT) and n(ALDH) could maintain over 75% of their activity under the same conditions. In addition, the solution of n(AOx-CAT) and n(ALDH) remained stable and free of aggregation in 2 weeks (FIG. 5). The increased stability would warrant the use of nanocapsules in vivo.

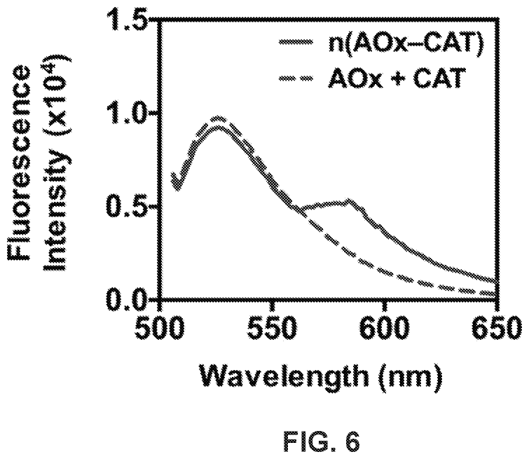

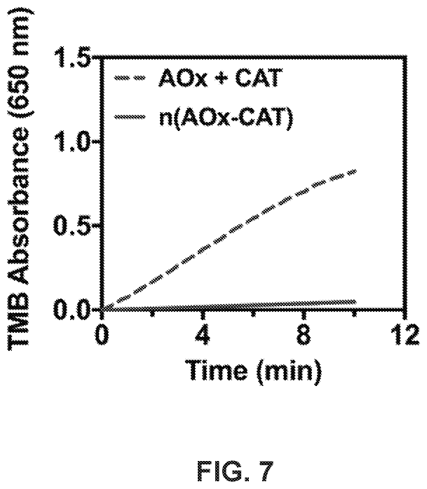

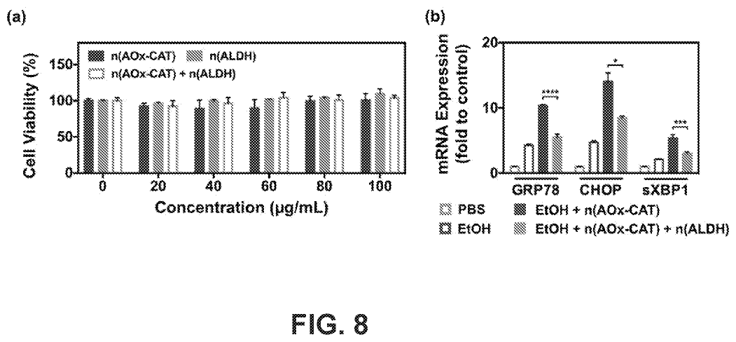

The close proximity of AOx and CAT within a nanocapsule was demonstrated using Förster resonance energy transfer (FRET), in which AOx and CAT were conjugated with fluorescein (FL) and tetramethylrhodamine (TAMRA), respectively (FIG. 6). Under 450 nm excitation, the mixture of AOx and CAT only exhibited an emission peak of FL at ˜520 nm. In contrast, n(AOx-CAT) showed emission peaks from both FL (520 nm) and TAMRA (580 nm), confirming the close association of the two enzymes in the nanocapsules. The close proximity of the AOx and CAT also enabled the efficient removal of the toxic H₂O₂ generated during the process of alcohol oxidation (FIG. 7). The effective breakdown of alcohol and acetaldehyde by the nanocapsules were confirmed by adding the two nanocapsules to an alcohol-containing solution (0.4%, w/v) (FIG. 2d). The concentration of ethanol continuously decreased (0.05% per hour), with only a small amount of acetaldehyde accumulated in the solution (0.006% per hour). Although n(AOx-CAT) and n(ALDH) were biocompatible, the acetaldehyde produced by n(AOx-CAT) during alcohol oxidation could induce severe cell injuries and apoptosis in primary mouse hepatocytes (PMH). The acetaldehyde produced by n(AOx-CAT) induced injuries among ˜36% of the cell population, while the addition of n(ALDH) substantially reduced the injury population to <6% (FIG. 2e). Furthermore, the cells treated with alcohol and n(AOx-CAT) showed a high-level of Caspase activity (3.0×10⁴ RLU), whereas adding n(ALDH) significantly decreased the level of Caspase (1.2×10⁴ RLU) (FIG. 2f, FIG. 8). The efficient and simultaneous breakdown of alcohol and acetaldehyde highlights the potential of co-delivering the two nanocapsules as an effective antidote for alcohol intoxication.

Synthesis of Enzyme Nanocapsules.

Native Alcohol oxidase (AOx) and Catalase (Cat) are first desalted to phosphate buffer (0.1M, pH 7.0). AOx is activated with 3-(2-pyridyldithio) propionic acid N-hydroxysuccinimide ester (SPDP) with a molar ratio of 10:1 (n/n, SPDP/AOx). The activation is performed for 2 hr at 4° C., following by dialysis against phosphate buffer (0.1M, pH=7). Cat is then activated with 2-iminothiolane hydrochloride. Reaction is performed at 4° C. for 2 h, following by dialysis against phosphate-EDTA buffer (0.1M phosphate, 1 mM EDTA, pH=7). Conjugation of AOx and Cat is then achieved by mixing equal mole of activated AOx and Cat (1:1, n/n) and incubated for 2 hr at 4° C. After conjugation, N-acryloxysuccinimide (NAS) was added into conjugated AOx-Cat solution (20:1, n/n, NAS/protein) to derive acryloxyl groups on the surface of enzymes. After dialysis against phosphate buffer (50 mM, pH 7.0), AOx-Cat solution was diluted to 1 mg protein/mL with phosphate buffer (50 mM, pH 7.0). Aldehyde hydrogenase (ALDH, ˜10 mg/mL) was dissolved in Tris buffer (50 mM, pH 8.0, 50 mM KCl) and passed through Zeba desalting column to remove the residual inorganic salts. Zinc acetate solution (final concentration 2 mM) was then added to block the active site of ALDH for 2 hr. Subsequently, the acryloyl groups were conjugated on ALDH with N-(3-aminopropyl) methacrylamide (APm)-modified succinimidyl 4-(N-maleimidomethyl) cyclohexane-1-carboxylate (SMCC), with a molar ratio of 15:1 (APm-SMCC:ALDH). After the conjugation reaction at 4° C. for 2 hr, EDTA (10 mM) was used to extract the zinc ions, followed by addition of 5,5-dithio-bis-(2-nitrobenzoic acid) (DTNB, Ellman's agent). After reacting for 15 min with DTNB, the modified ALDH was passed through Zeba desalting column to remove the excess small molecules.

The AOx-CAT or ALDH nanocapsules are then prepared via in situ polymerization using acrylamide (AAm), APm, and N,N′-methylenebisacrylamide (BIS) as the monomer and crosslinker, and ammonium persulfate (APS) and N,N,N′,N′-tetramethylethylenediamine (TEMED) as the initiator. The polymerization reaction is continued at 4° C. for 1 hr before the reaction mixture is dialyzed in phosphate buffer to remove unreacted small molecules. The resulting enzyme nanocapsules are termed as n(AOx-CAT) and n(ALDH), respectively.

For n(ALDH), an additional step of tris-(2-carboxyethyl) phosphine (TCEP, 10 mM, pH 7.0) treatment is used to reduce the disulfide bonds. The active n(ALDH) is then passed through the desalting column to exchange to potassium phosphate buffer (50 mM, pH 8.0, 50 mM NaCl).

Morphology, Activity, and Biocompatibility of Enzyme Nanocapsules.

Spherical and monodispersed n(AOx-CAT) and n(ALDH) averaging 32.8±4.0 nm and 34.3±3.9 nm were observed with transmission electron microscopy and dynamic light scattering (FIG. 2a, b). Meanwhile, n(AOx-CAT) and n(ALDH) showed zeta potentials of ˜4 mV and ˜2 mV, respectively (FIG. 2c). The positive zeta potentials would allow their rapid accumulation in the liver after administration. While the native enzymes are found to be unstable under physiological temperature or in the presence of proteases, the polymer shells also enhance the thermal and proteolytic stability of the enzymes. For instance, when incubated at 37° C. for 2 hr, especially in the presence of protease, the native enzymes quickly lost their activity. On the contrary, both n(AOx-CAT) and n(ALDH) could maintain over 75% of their activity under the same conditions. In addition, the solution of n(AOx-CAT) and n(ALDH) remained stable and free of aggregation in 2 weeks. The increased stability would warrant the use of nanocapsules in vivo.

The effective breakdown of alcohol and acetaldehyde by the nanocapsules were confirmed by adding the two nanocapsules to an alcohol-containing solution (0.4%, w/v) (FIG. 2d). The concentration of ethanol continuously decreased (0.05% per hour), with only a small amount of acetaldehyde accumulated in the solution (0.006% per hour). Although n(AOx-CAT) and n(ALDH) were biocompatible, the acetaldehyde produced by n(AOx-CAT) during alcohol oxidation could induce severe cell injuries and apoptosis in primary mouse hepatocytes (PMH). The acetaldehyde produced by n(AOx-CAT) induced injuries among ˜36% of the cell population, while the addition of n(ALDH) substantially reduced the injury population to <6% (FIG. 2e). Furthermore, the cells treated with alcohol and n(AOx-CAT) showed a high-level of Caspase activity (3.0×10⁴ RLU), whereas adding n(ALDH) significantly decreased the level of Caspase (1.2×10⁴ RLU) (FIG. 2f). The efficient and simultaneous breakdown of alcohol and acetaldehyde highlights the potential of co-delivering the two nanocapsules as an effective antidote for alcohol intoxication.

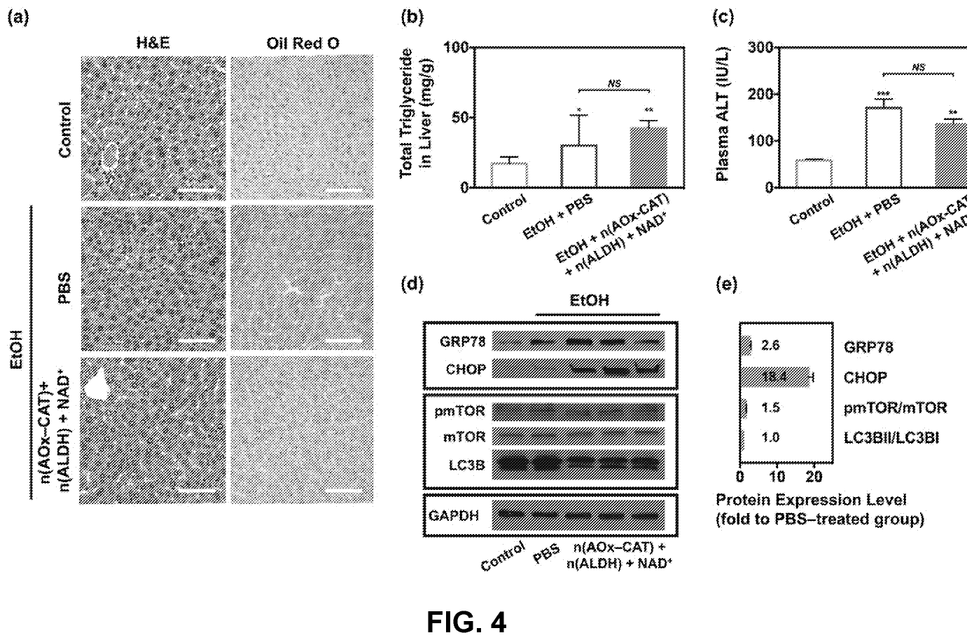

To evaluate the organelle stress responses in the liver, we investigated the expression levels of ER stress markers (GRP78, CHOP) and autophagy markers (pmTOR, mTOR, LC3B) (FIG. 4d). Compared with the PBS-treated group, the expression levels of GRP78, CHOP, pmTOR/mTOR, and LC3BII/LC3BI in the antidote-treated group were upregulated 2.6, 18.4, 1.5, and 1.0-fold, respectively. All these markers but CHOP indicated negligible organelle stress responses and autophagy disruptions. With regards to CHOP in this chronic experimental system, the complete elimination of alcohol and acetaldehyde with even faster kinetics would potentially reduce its expression level and achieve complete liver protection. Collectively, the antidote allows the efficient removal of both alcohol and acetaldehyde, without significant disruption to the liver health.

Delivery and Efficacy of the Antidote.

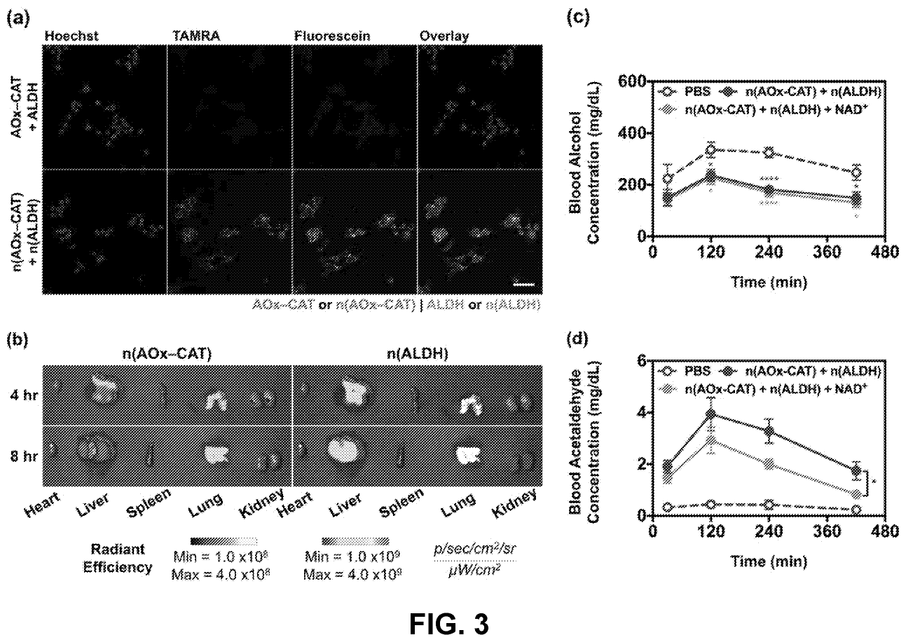

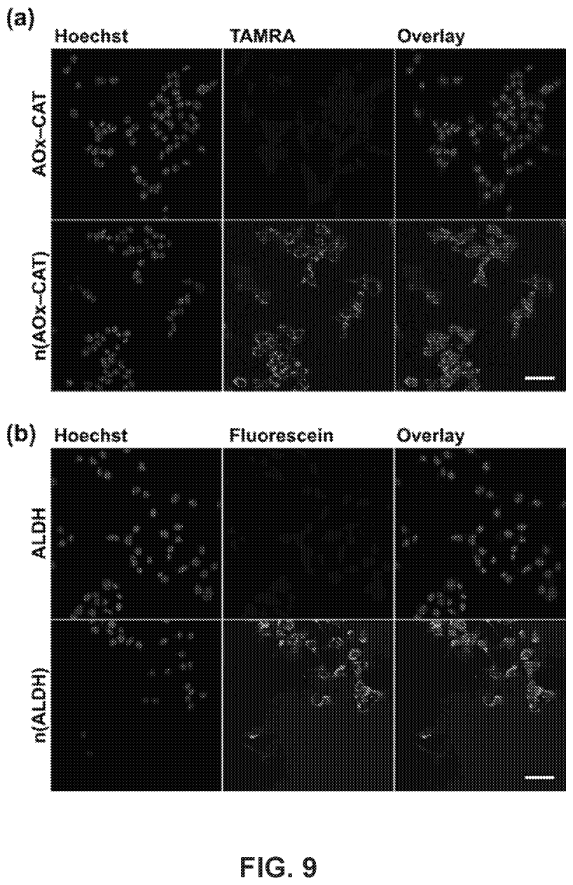

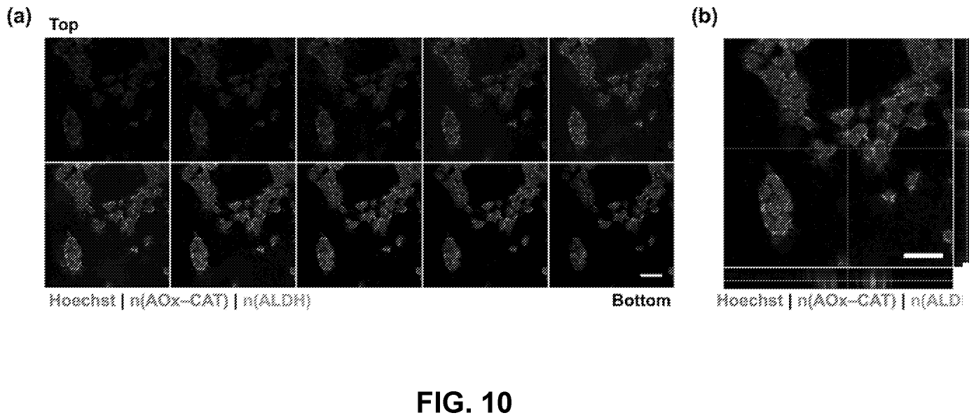

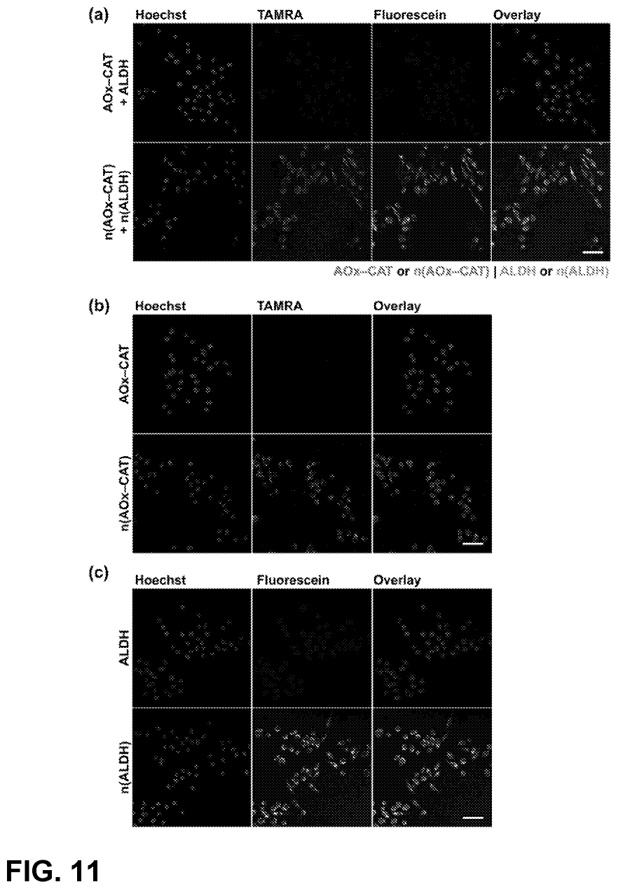

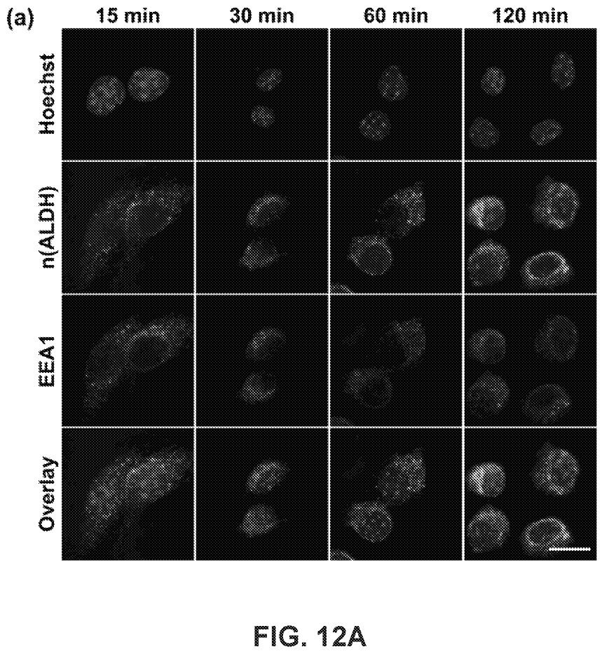

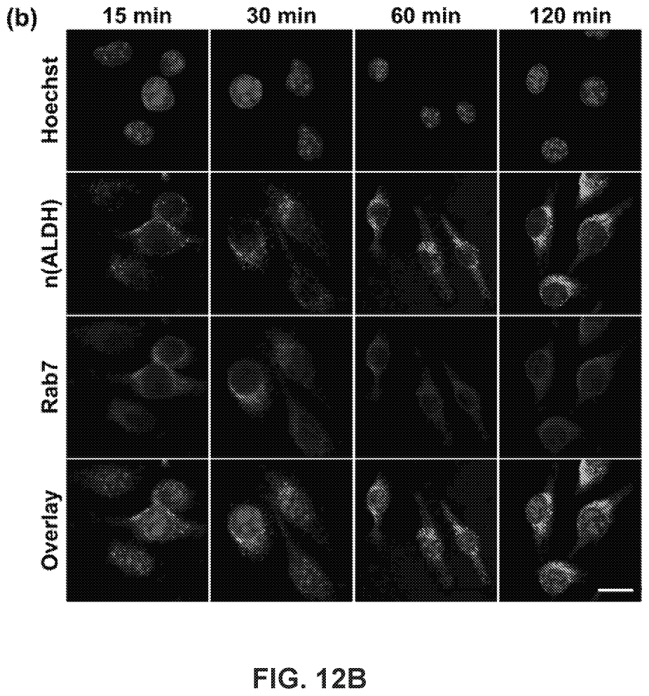

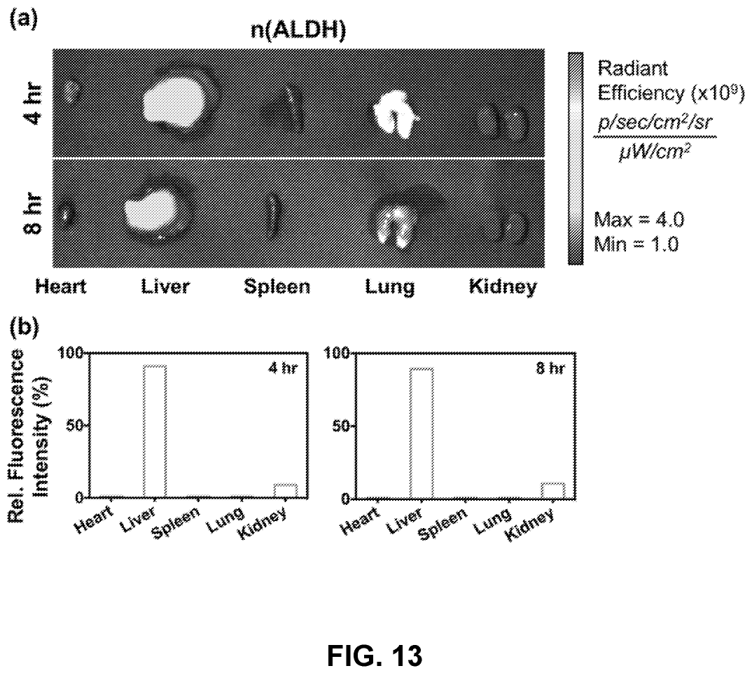

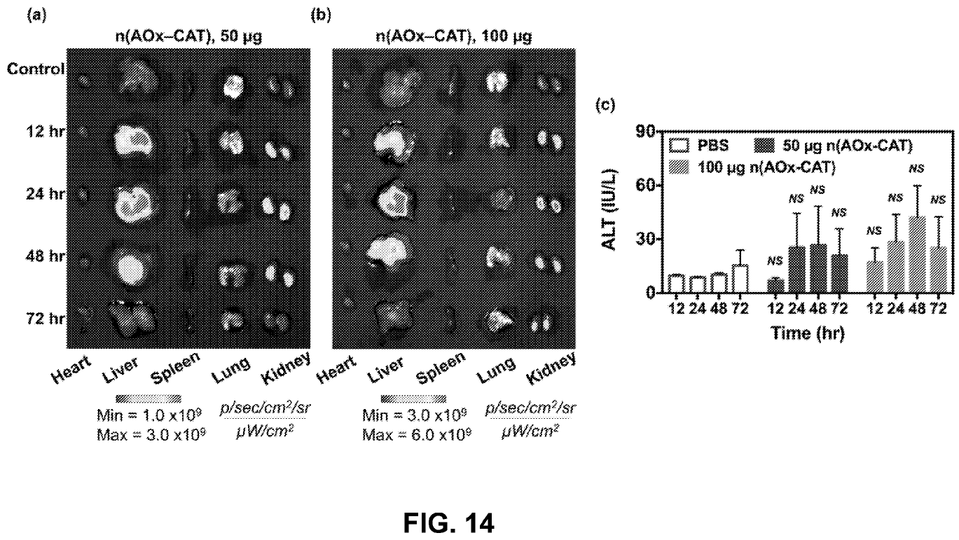

Similar to other positively-charged nanoparticles, intravenous administration of the nanocapsules enables their accumulation in the liver^{[23,24,33]}, the major organ for alcohol metabolism. To confirm their effective delivery to the liver, we first examined the uptake of n(AOx-CAT) and n(ALDH) by hepatocytes (FIG. 3a, FIG. 9). Herein, the native AOx-CAT and n(AOx-CAT) were conjugated with TAMRA, and the native ALDH and n(ALDH) were conjugated with FL. After incubation with mouse hepatocytes (AML12) for 4 hr, the cells treated with the native AOx-CAT and ALDH exhibited little fluorescence, whereas intense fluorescence signals were observed from the cells incubated with n(AOx-CAT) and n(ALDH). Moreover, the fluorescence signals from n(AOx-CAT) and n(ALDH) overlapped in the cytosol of the hepatocytes^{[19,35]}, indicating the co-delivery of the two nanocapsules to the same cells (FIG. 10). Similar results were also observed in mouse macrophages (J774A.1), which could transport the nanocapsules from the circulation to the liver (FIG. 11). With both n(AOx-CAT) and n(ALDH) internalized in the cytosol through endocytosis (FIG. 12), these cells can function as mini-reactors to eliminate alcohol and acetaldehyde simultaneously. The biodistribution of the nanocapsules in mice was further investigated with n(AOx-CAT) and n(ALDH) conjugated with TAMRA and Alexa Fluor 680 (AF680), respectively. The nanocapsules were intravenously administered to the mice, and the organs were imaged 4 and 8 hr post-injection (FIG. 3b, FIG. 13). High TAMRA and AF680 intensities were observed predominantly in the liver, indicating the efficient delivery of both nanocapsules to the liver. The rapid accumulation of n(AOx-CAT) and n(ALDH) would potentially aid in the consecutive breakdown of alcohol and acetaldehyde. To investigate the potential secondary poisoning that may be caused by the degradation of the nanocapsules, we administered the n(AOx-CAT) (as an example of nanocapsules) to the mice to study their biodistribution. From fluorescence imaging, we observed that most of the nanocapsules rapidly accumulated in the liver and the fluorescence intensity gradually decreased in the next 3 days. Only slight increases in the ALT levels during the first 48 hr after the administration of the nanocapsules were observed. (FIG. 14).

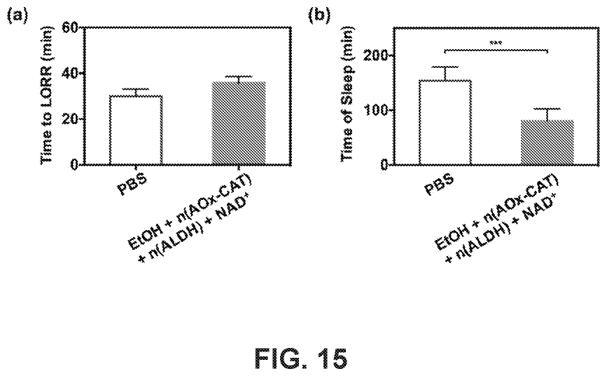

To study the efficacy of the nanocapsules as an antidote, we intravenously administered n(AOx-CAT) and n(ALDH) with or without additional NAD⁺ to the alcohol-intoxicated mice (5 mg alcohol per gram of mouse body weight). Additional NAD⁺ was used to evaluate if acetaldehyde oxidation by n(ALDH) could be enhanced. The blood samples were taken at different time after the administration (30, 120, 240, and 420 min) to determine the BAC and blood acetaldehyde concentrations (BAchC). Compared to the PBS-treated group that showed a BAC of ˜335, ˜325, and ˜250 mg/dL at 120, 240, and 420 min, the group treated with nanocapsules (without NAD⁺) showed a BAC of ˜236, ˜182, and ˜127 mg/dL, respectively (FIG. 3c). The group given the nanocapsules with NAD⁺ exhibited a similar BAC to the group given nanocapsules alone, suggesting that the alcohol oxidation by n(AOx-CAT) was independent of the level of NAD⁺. The substantial decrease in BAC demonstrates the efficacy of the nanocapsules as an antidote and results in a faster restoration of consciousness (FIG. 15). More importantly, the acetaldehyde generated from alcohol oxidation by n(AOx-CAT) could be rapidly eliminated by n(ALDH). In the group given nanocapsules (without NAD⁺), the BAchC remained at ˜4.0, ˜3.3, and ˜1.9 mg/dL at 120, 240, and 420 min (FIG. 3d). Moreover, the additional NAD⁺ could help further decrease the BAchC to ˜3.0, ˜2.0, and ˜0.8 mg/dL at 120, 240, and 420 min. The extremely low BAchC would significantly contribute to the liver protection, given that the accumulation of acetaldehyde could induce liver cirrhosis and hepatocellular carcinoma^{[17,36-39]}. The simultaneous and efficient removal of both alcohol and acetaldehyde highlighted the feasibility of using n(AOx-CAT) and n(ALDH) as an antidote toward alcohol intoxication or poisoning.

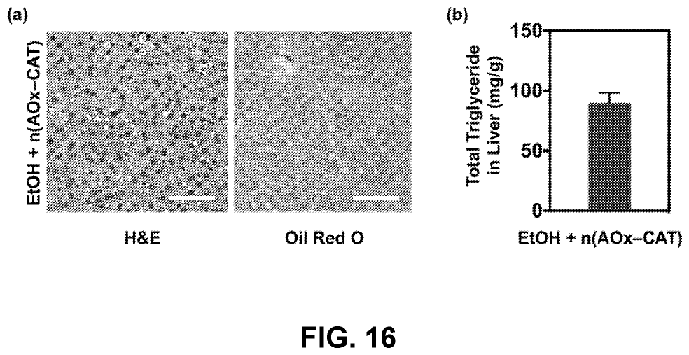

While acute alcohol intoxication causes mild elevation of ALT and steatosis, liver injury becomes more evident with chronic high-fat diet (HFD) plus a single binge^{[40]}. Thus, we studied the alcohol-induced liver injury and organelle stress response in mice given HFD for 3 weeks, followed by acute alcohol intoxication. The mice were then treated with PBS, or n(AOx-CAT) and n(ALDH) with NAD⁺ as the antidote, and their liver samples were analyzed. Compared with the healthy liver, the formation of lipid droplets (LD) was slightly increased in alcohol-intoxicated mice given PBS or the antidote (FIG. 4a). Consistent with the histology, the liver triglyceride content was 30 and 42 mg/g in the group treated with PBS and the antidote, respectively (FIG. 4b). While the accumulation of acetaldehyde in the liver of mice treated only with n(AOx-CAT) could substantially increase LD formation (FIG. 16), the efficient removal of acetaldehyde by the antidote reduced it remarkably. Moreover, the plasma ALT level was increased 170 IU/L after alcohol intake, whereas the antidote brought the level down to 135 IU/L (FIG. 4c). Although the administration of the antidote exhibited a higher level of liver triglyceride and ALT than those of the healthy mice, BAC and BAchC were significantly decreased, and sufficient liver protection was achieved.

To evaluate the organelle stress responses in the liver, we investigated the expression levels of ER stress markers (GRP78, CHOP)^{[36,41,42]} and autophagy markers (pmTOR, mTOR, LC3B)^{[43]} (FIG. 4d). Compared with the PBS-treated group, the expression levels of GRP78, CHOP, pmTOR/mTOR, and LC3BII/LC3BI in the antidote-treated group were upregulated 2.6, 18.4, 1.5, and 1.0-fold, respectively. All these markers but CHOP indicated negligible organelle stress responses and autophagy disruptions. With regards to CHOP in this chronic experimental system, the complete elimination of alcohol and acetaldehyde with even faster kinetics would potentially reduce its expression level and achieve complete liver protection. Collectively, the antidote allows the efficient removal of both alcohol and acetaldehyde, without significant disruption to the liver health.

### Examples

### Example 1. Synthesis of Enzyme Nanocapsules

All the enzyme nanocapsules were prepared one day before the animal experiments. Alcohol oxidase (AOx) and Catalase (CAT) dual-enzyme nanocapsules were prepared as previously described (see, e.g. Y. Liu et al., Nat. Nanotechnol. 2013, 8, 187). Synthesis of aldehyde dehydrogenase (ALDH) nanocapsule is demonstrated in FIG. 1b. In detail, ALDH (˜10 mg/mL, purchased from MP Biomedicals) was dissolved in Tris buffer (50 mM, pH 8.0, 50 mM KCl) and passed through Zeba desalting column (Thermo-Fisher Scientific) to remove the residual inorganic salts. Zinc acetate solution (final concentration 2 mM) was then added to block the active site of ALDH for 2 hr. Subsequently, the acryloyl groups were conjugated on ALDH with N-(3-aminopropyl) methacrylamide (APm)-modified succinimidyl 4-N-maleimidomethyl) cyclohexane-1-carboxylate (SMCC), with a molar ratio of 15:1 (APm-SMCC: ALDH). After the conjugation reaction at 4° C. for 2 hr, EDTA (10 mM) was used to extract the zinc ions, followed by addition of 5,5-dithio-bis-(2-nitrobenzoic acid) (DTNB, Ellman's agent). After reacting for 15 min with DTNB, the modified ALDH was passed through Zeba desalting column to remove the excess small molecules. The ALDH nanocapsules were then prepared via in situ polymerization using acrylamide (AAm, 6000:1, n/n, AAm:ALDH), APm (100:1, nh, APm:ALDH), and N,N′-methylenebisacrylamide (BIS, 1000:1, nn, AAm:ALDH) as the monomer and crosslinker, and ammonium persulfate (APS, 500:1, nn, APS:ALDH) and N,N,N′,N-tetramethylethylenediamine (TEMED, 2:1, w-w, TEMED:APS) as the initiator. The polymerization reaction was continued at 4° C. for 1 hr before the reaction mixture was dialyzed in Tris buffer to remove unreacted small molecules. To synthesize nanocapsules with higher zeta potentials, additional APm was added to the polymerization mixture. In addition, tris-(2-carboxyethyl) phosphine (TCEP, 10 mM, pH 7.0) solution was used to reduce the disulfide bonds. The active n(ALDH) was then passed through the desalting column to exchange to potassium phosphate buffer (50 mM, pH 8.0, 50 mM NaCl). Synthesized n(ALDH) was purified with an ion-exchange column (Q Sepharose Fast Flow, GE Healthcare) to exclude the un-encapsulated ALDH. The purified n(ALDH) was stored at −80° C. for later experiments.

### Example 2: Enzyme Activity Assays

The native AOx-CAT and n(AOx-CAT) were dissolved in a solution containing HEPES (50 mM, pH 7.0) and alcohol (0.1%, w/v). The reaction for alcohol oxidation was carried out at room temperature for 5 min and the generation of acetaldehyde was measured based on its reaction with 3-methyl-2-benzothiazolinone hydrazine (MBTH). In brief, one volume of the acetaldehyde standard (Sigma Aldrich, ACS grade) or the sample was mixed with one volume of 0.8% (w/v) MBTH. Meanwhile, another one volume of 0.8% (w/v) MBTH was mixed with 1% (w/v) iron(III) chloride. The two solutions were incubated at room temperature for 15 min and equally mixed. The blue color that MBTH-acetaldehyde complex formed immediately after mixing was measured with a spectrophotometer at 600 nm. A standard curve with different acetaldehyde concentrations (250, 125, 62.5, 32.2, 15.6, 7.8 ppm) was prepared as a reference. The change in A600 was proportional to the activity of AOx-CAT.

The native ALDH and n(ALDH) were dissolved in a solution containing Tris-HCl (100 mM, pH 8.0), KCl (300 mM), acetaldehyde (160 μM), 2-mercaptoethanol (10 mM) and NAD⁺ (20 mM). The reaction for acetaldehyde degradation was carried out at room temperature for 5 min and the absorbance at 340 nm (A340) was recorded by a spectrophotometer. The change in A340 which was proportional to the residual activity of ALDH was recorded. The conversion of NAD⁺ to NADH per minute and the percentage of residual activity relative to the native ALDH were then calculated.

### Example 3: Stability Assays

Thermal stability was conducted by incubating the native enzymes (AOx-CAT or ALDH) and nanocapsules (n(AOx-CAT) or n(ALDH)) (0.1 mg/mL) at 37° C. for 2 hr. Samples were taken at different time, and the residual activity was determined with activity assays. Proteolytic stability included trypsin (0.2 mg/mL) in each mixture during incubation, and the rest of the measurements were the same as in the thermal stability measurements. Long-term stability was performed by monitoring the size of n(AOx-CAT) and n(ALDH) for 2 weeks. Nanocapsules were maintained in PBS (pH 7.4) at 4° C. during the 2-week period.

### Example 4: Characterization of Enzyme Nanocapsules

The morphology of n(AOx-CAT) and n(ALDH) was observed by Transmission Electron Microscopy (TEM). TEM samples were prepared by pipetting 2 μL nanocapsules to a carbon-coated copper grid. The droplet of the nanocapsules was in contact with the grid for 1 min, before rinsing with water and staining with 1% (w/v) sodium phosphotungstate (pH 7.0) for 30 s. Dynamic Light Scattering (DLS) measurements were conducted on a Malvern Zetasizer Nano instrument. The number distribution and zeta potential of the nanocapsules were measured at 1.0 mg/mL in phosphate buffer (10 mM, pH 7.0). The Forster resonance energy transfer (FRET) in n(AOx-CAT) or the mixture of AOx and CAT was measured with a plate reader (M200, Tecan), with an excitation wavelength of 450 nm.

### Example 5: Kinetics of H₂O₂ Generation

The generation of H₂O₂ was measured using horseradish peroxidase and 3,3′,5,5′-tetramethylbenzidine (HRP/TMB) assay. HRP, TMB, and alcohol were added to the mixture to a final concentration of 1 μg/mL, 1 mg/mL, and 1 mg/mL, respectively. The reaction was initiated by the addition of AOx-CAT or the mixture of AOx and CAT. The change in A650 was recorded with a spectrophotometer.

### Example 6: Measurement of Alcohol and Acetaldehyde Concentrations

Blood samples were taken at different time points and centrifuged at 2000×g for 10 min twice. The supernatant (plasma) was collected and used for further measurements. The measurement of blood alcohol concentration has been described previously. Blood acetaldehyde concentration was measured based on its reaction with MBTH described above. The exact concentration of acetaldehyde in the samples was referred to the standard curve.

### Example 7. Cell Culture

HeLa, AML12, and J774A.1 cells were purchased from American Type Culture Collection (ATCC). HeLa cells were cultured on 25 cm² tissue culture flasks (Thermo-Fisher Scientific) and maintained by Eagle's Minimum Essential Medium (EMEM), supplemented with 10% fetal bovine serum (FBS) and 1% penicillin/streptomycin (P/S). AML12 and J774A.1 cells were cultured under the same condition but with Dulbecco's Modified Eagle Media (DMEM). The primary mouse hepatocytes were isolated by USC Liver Cell Culture Core. The isolated cells were allowed for attachment by 4 hr and the medium was switched to William's E medium (Thermo-Fisher Scientific) supplemented with dexamethasone, insulin, transferrin, sodium selenium, reduced FBS, GlutMax and P/S. The primary cells were allowed to stay at 37° C. and 5% CO₂ overnight. On the next day, the cells were treated with alcohol and/or the nanocapsules. After the treatments, the cells were washed with ice-cold PBS and subjected to protein and RNA extractions. All in vitro assays were repeated at least three times for each measurement.

### Example 8: Cell Viability Assays

In HeLa cells, cell viability was quantified with CellTiter Blue Assay Kit (Promega). The live cells effectively convert the non-fluorescent resazurin to the fluorescent resorufin (Ex.=560 nm, Em.=590 nm). Cell viability was measured on a TECAN microplate reader. To assess the cytotoxicity in the primary mouse hepatocytes (PMH), the release of lactate dehydrogenase (LDH) into extracellular space was measured. LDH is enriched in the cytoplasm of PMH and its release into the culture medium indicates the loss of membrane integrity. The amount of LDH in the medium that is proportional to the number of dead cells was measured by Pierce™ LDH Cytotoxicity Assay Kit (Thermo-Fisher Scientific) according to manufacturer's instructions and quantified by creating a standard curve with a known number of cells. Induction of apoptosis was evaluated by the Caspase activity in alcohol-treated cells. Effector Caspase 3/7 activity was measured with Caspase-Glo® 3/7 assay system (Promega) according to manufacturer's instructions. The activity of effector Caspases was indicated by relative luminescent unit (RLU) measured by an Omega microplate reader.

### Example 9: Immunoblotting and qPCR

Extraction of protein and RNA, immunoblotting and qPCR were described previously (see, e.g. H. Han et al., Hepatol. Commun. 2017, 1, 122). Primary antibodies for GRP78, LC3B, mTOR, pmTOR, CHOP and secondary antibodies were purchased from Cell Singling Corp. Primers of ER stress markers were selected according to art accepted practices.

### Example 10: Cellular Uptake Experiment

Hepatocyte (AML12) and macrophage (J774A.1) uptake of the nanocapsules were studied using confocal laser scanning microscopy (CLMS). Cells were seeded in 8-well chambers (ibidi) pretreated with Cell-Tak (Corning) one day before the experiment. AML12 and J774A.1 were incubated with the native enzymes or nanocapsules at 0.5 mg/mL for 4 hr at 37° C., and then washed extensively with FluoroBrite DMEM Media (Gibco) to remove the residual culture media. Nuclei were stained with Hoechst 33342 and the cells were observed with inverted Leica TCS-SP8-SMD confocal microscope.

J774A.1 cells were used to study the trafficking of nanocapsules. After incubation with n(ALDH) for 15, 30, 60, and 120 min, J774A.1 cells were washed, fixed with 4% paraformaldehyde, permeated with 1% Triton X-100 (Sigma Aldrich), blocked with 5% BSA, and treated with rabbit anti-EEA1 antibody (Cell Signaling Corp.) or rabbit anti-Rab7 antibody (Cell Signaling Corp.) overnight. Cells were then stained with goat anti-rabbit IgG (Alexa Fluor 594, Abcam) and nuclei were stained with Hoechst 33342. Cells were observed with confocal microscope.

### Example 11: Biodistribution of Nanocapsules

All animals were treated in accordance with the Guide for Care and Use of Laboratory Animals and the study was approved by the local animal care committee. The biodistribution of nanocapsules in mice were studied using fluorescence imaging (IVIS Lumina II, Perkin Elmer). n(AOx-CAT) and n(ALDH) were labeled with TAMRA and Alexa Fluor 680 (AF680), respectively. Single nanocapsules exemplified by n(ALDH) or both n(AOx-CAT) and n(ALDH) were intravenously injected to mice via tail vein at a dosage of 100 μL (1 mg/mL) per animal. Mice were sacrificed 4 hr and 8 hr post-injection, and major organs were collected for fluorescence imaging.

### Example 12. In Vivo Biocompatibility

The biodistribution of nanocapsules in mice were studied using fluorescence imaging (IVIS Lumina II, Perkin Elmer). n(AOx-CAT) was labeled with Alexa Fluor 680 (AF680) and used as an example of the nanocapsules. n(AOx-CAT) was intravenously injected to mice via tail vein at a dosage of 50 or 100 μL (1 mg/mL) per animal. Mice were sacrificed 12, 24, 48, and 72 hr post-injection, and major organs were collected for fluorescence imaging. The liver samples from mice given non-labeled n(AOx-CAT) were collected for liver toxicity assessment. The liver samples were rinsed extensively in PBS, and then homogenized with Bead Mill 24 Homogenizer (Thermo-Fisher Scientific). The supernatant of the homogenate after centrifugation (10,000×g, 15 min, 4° C.) was collected and used for the ALT assay. The liver ALT was evaluated with Alanine Transaminase Colorimetric Activity Assay Kit (Cayman Chemical) according to manufacturer's instructions. The ALT activity was measured with a Tecan microplate reader.

### Example 13: Animal Experiments and Loss of the Righting Reflex Assay

Male C57BL/6 mice were purchased from the Jackson Laboratory. Loss of the righting reflex (LORR) assay has been used to assess and quantify the functional tolerance and consciousness in acute drinking models (see, e.g. S. Perreau-Lenz et al., Addict. Biol. 2009, 14, 253). In brief, mice were gavaged with 30% alcohol in normal saline (5 mg/g body weight) or the same amount of isocaloric maltose solution as the control. Mice were subsequently injected with 50 μg of n(AOx-CAT) and/or 0.5 mg of n(ALDH). The solution used to dissolve the nanocapsules containing NAD⁺ was injected as the control. The mice were then placed in a cylinder rotated for 90° for every 2 sec to determine the time of LORR at which mice stopped flipping from a supine position within 5 sec after rotation. After that, the mice were tested every 10 min for recovery from LORR. The period between LORR and recovery from LORR was defined as the time of sleep for this study. Mice were sacrificed at 8 hr for further analysis.

### Example 14: Chronic Alcohol Feeding and Liver Pathology

Mice were given high-fat diet (HFD) for 21 days. On the 21^{st} day, mice were starved for ˜12 hr and gavaged with 30% alcohol in PBS (5 mg/g body weight) or the same volume of isocaloric maltose solution as the control. Mice were injected with 50 μg of n(AOx-CAT) and/or 0.5 mg of n(ALDH) within 30 min after the alcohol gavage. The solution used to dissolve the nanocapsules containing NAD⁺ was injected as the control. The mice were sacrificed after 8 hr for the following analyses. Plasma alanine aminotransferase (ALT) and total liver triglyceride were measured as described previously (see, e.g. H. Han et al., Hepatol. Comnun. 2017, 1, 122). For hematoxylin and eosin staining (H&E), liver tissues were fixed in 10% formalin overnight at 4° C., washed with and stored in 80% alcohol. The fixed tissues were embedded in paraffin, sectioned at 5 μm and proceeded to H&E. For Oil Red O staining, liver tissues were embedded in O.C.T. (Sakura® Finetek), snap-frozen, sectioned at 5 μm and mounted on glass slides. The tissues on the slides were fixed in 10% formalin and stained with an Oil Red O isopropanol solution (Electron Microscopy Sciences, Hatfield, Pa.).

### Example 15: Statistics

Data are presented as means SEM unless otherwise indicated. Statistical analyses were performed with GraphPad Prism® 6 using the one way-ANOVA for comparison of multiple groups and two-way ANOVA for comparison of trends between different treatments. The P values of 0.05 or less are considered significant.

### REFERENCE

[1] L. Gunzerath, B. G. Hewitt, T.-K. Li, K. K Warren, Ann. N. Y. Acad. Sci. 2011, 1216, 1.
[2] P. D. Friedmann, N. Engl. J. Med. 2013, 368, 365.
[3] W. Gilmore, T. Chikritzhs, T. Stockwell, D. Jernigan, T. Naimi, I. Gilmore, Nat. Rev. Gastroenterol. Hepatol. 2016, 13, 426.
[4] K. Lee, L. Møller, F. Hardt, A. Haubek, E. Jensen, The Lancet 1979, 2, 759.
[5] J. Fernández-Solà, Nat. Rev. Cardiol. 2015, 12, 576.
[6] J. I. Garaycoechea, G. P. Crossan, F. Langevin, L. Mulderrig, S. Louzada, F. Yang, G. Guilbaud, N. Park, S. Roerink, S. Nik-Zainal, M. R. Stratton, K. J. Patel, Nature 2018, 553, 171.
[7] M. Moss, E. L. Burnham, The Lancet 2006, 368, 2231.
[8] C. Jamaty, B. Bailey, A. Larocque, E. Notebaert, K. Sanogo, J.-M. Chauny, Clin. Toxicol. 2010, 48, 1.
[9] N. Bertrand, C. Bouvet, P. Moreau, J.-C. Leroux, ACS Nano 2010, 4, 7552.
[10] Y. Shen, A. K. Lindemeyer, C. Gonzalez, X. M. Shao, I. Spigelman, R. W. Olsen, J. Liang, J. Neurosci. 2012, 32, 390.
[11] J. P. Connor, P. S. Haber, W. D. Hall, The Lancet 2016, 387,988.
[12] F. Wang, Y. Li, Y.-J. Zhang, Y. Zhou, S. Li, H.-B. Li, Molecules 2016, 21, 64.
[13] H. J. Hanchar, P. Chutsrinopkun, P. Meera, P. Supavilai, W. Sieghart, M. Wallner, R. W. Olsen, Proc. Natl. Acad. Sci. USA 2006, 103, 8546.
[14] J. Liang, R W. Olsen, Acta Pharmacol Sin 2014, 35, 981.
[15] A. Sun, L. Mu, X. Hu, ACS Appl. Mater. Interfaces 2017, 9,12241.
[16] X. Qu, M. Gou, J. Zaidan, K. Zhang, S. Chen, Nanomedicine. 2014, 9, 2437.
[17] C. S. Lieber, Drug Alcohol Rev. 1991, 10, 175.
[18] S. Zakhari, Alcohol Res. Health 2006, 29, 245.
[19] M. Yan, J. Du, Z. Gu, M. Liang, Y. Hu, W. Zhang, S. Priceman, L. Wu, Z. H. Zhou, Z. Liu, T. Segura, Y. Tang, Y. Lu, Nat. Nanotechnol. 2010, 5, 48.
[20] Y. Liu, J. Du, M. Yan, M. Y. Lau, J. Hu, H. Han, O. O. Yang, S. Liang, W. Wei, H. Wang, J. Li, X. Zhu, L. Shi, W. Chen, C. Ji, Y. Lu, Nat. Nanotechnol. 2013, 8, 187.
[21] S. Liang, Y. Liu, X. Jin, G. Liu, J. Wen, L. Zhang, J. Li, X. Yuan, I. S. Y. Chen, W. Chen, H. Wang, L. Shi, X. Zhu, Y. Lu, Nano Res. 2016, 9, 1022.
[22] X. Zhang, D. Xu, X. Jin, G. Liu, S. Liang, H. Wang, W. Chen, X. Zhu, Y. Lu, J. Control. Release 2017, 255, 54.
[23] K. M. Tsoi, S. A. MacParland, X.-Z. Ma, V. N. Spetzler, J. Echeverri, B. Ouyang, S. M. Fadel, E. A. Sykes, N. Goldaracena, J. M. Kaths, J. B. Conneely, B. A. Alman, M. Selmer, M. A. Ostrowski, O. A. Adeyi, A. Zilman, I. D. McGilvray, W. C. W. Chan, Nat. Mater. 2016, 15, 1212.
[24] E. Blanco, H. Shen, Mi Ferrari, Nat. Biotechnol. 2015, 33, 941.
[25] Y. Lu, A. A. Aimetti, R Langer, Z. Gu, Nat. Rev. Mater. 2016, 1, 16075.
[26] M. Magnani, M Laguerre, L. Rossi, M Bianchi, P. Ninfali, F. Mangani, C. Ropars, Alcohol Alcohol. 1990, 25, 627.
[27] C. Lizano, S. Sanz, J. Luque, M. Pinilla, Biochim. Biophys. Acta 1998, 1425, 328.
[28] C. Lizano, M T. Pérez, M. Pinilla, Life Sci. 2001, 68, 2001.
[29] A. D'Alessandro, G. Liumbruno, G. Grazzini, L. Zolla, Blood Transfus. 2010, 8, 82.
[30] M. Garcia-Roa, M. Del Carmen Vicente-Ayuso, A. M. Bobes, A. C. Pedraza, A. González-Fernndez, M. P. Martin, I. Saez, J. Seghatchian, L. Gutirrez, Blood Transfius. 2017, 15, 222.
[31] S. Hart, C. M. Cserti-Gazdewich, S. A. McCluskey, Anaesthesia 2015, 70, 38.
[32] A. I. Cederbaum, Clin. Liver Dis. 2012, 16, 667.
[33] S. G. Elci, Y. Jiang, B. Yan, S. T. Kim, K. Saha, D. F. Moyano, G. Yesilbag Tonga, L. C. Jackson, V. M. Rotello, R. W. Vachet, ACS Nano 2016, 10, 5536.
[34] F. Alexis, E. Pridgen, L. K. Molnar, O. C. Farokhzad, Mol. Pharm. 2008, 5, 505.
[35] W. Sun, T. Jiang, Y. Lu, M. Reiff, R. Mo, Z. Gu, J. Am. Chem. Soc. 2014, 136, 14722.
[36] H. K. Seitz, F. Stickel, Nat. Rev. Cancer 2007, 7, 599.
[37] M. Setshedi, J. R. Wands, S. M. de la Monte, Oxid. Med. Cell. Longev. 2010, 3, 178.
[38] P. J. Brooks, J. A. Theruvathu, Alcohol 2005, 35, 187.
[39] R. Parker, S. J. Kim, B. Gao. Nat. Rev. Gastroenterol. Hepatol. 2018, 15, 50.
[40] B. Chang, M.-J. Xu, Z. Zhou, Y. Cai, M. Li, W. Wang, D. Feng, A. Bertola, H. Wang, G. Kunos, B. Gao, Hepatology 2015, 62, 1070.
[41] C. Ji, Biomolecules 2015, 5, 1099.
[42] C. Ji, N. Kaplowitz, M. Y. Lau, E. Kao, L. M. Petrovic, A. S. Lee, Hepatology 2011, 54, 229.
[43] W.-X. Ding, S. Manley, H.-M. Ni, Exp. Biol. Med. 2011, 236, 546.

All publications mentioned herein (e.g. those above, Xu et al., Adv Mater. 2018 May; 30(22):e1707443; U.S. Pat. No. 10,016,490, U.S. application Ser. No. 15/531,356; and U.S. Patent Publications US-2014-0134700 and US-2014-0186436) are incorporated herein by reference to disclose and describe the methods and/or materials in connection with which the publications are cited. Publications cited herein are cited for their disclosure prior to the filing date of the present application. Nothing here is to be construed as an admission that the inventors are not entitled to antedate the publications by virtue of an earlier priority date or prior date of invention. Further, the actual publication dates may be different from those shown and require independent verification.

### CONCLUSION

This concludes the description of the illustrative embodiments of the present invention. The foregoing description of one or more embodiments of the invention has been presented for the purposes of illustration and description. It is not intended to be exhaustive or to limit the invention to the precise form disclosed. Many modifications and variations are possible in light of the above teaching.

1. A method of decreasing the concentration of ethanol and its metabolites in an individual comprising the steps of:
(a) administering a multiple-enzyme nanocomplex system to the individual, wherein the multiple-enzyme nanocomplex system comprises:
an alcohol oxidase enzyme that generates hydrogen peroxide and acetaldehyde a first enzymatic reaction with ethanol;
a catalase enzyme that converts the hydrogen peroxide into water in a second enzymatic reaction; and
a polymeric network configured to form a shell that encapsulates the alcohol oxidase and the catalase, wherein:
the polymeric network exhibits a permeability sufficient to allow the ethanol to diffuse from an external environment outside of the shell to the alcohol oxidase so that the hydrogen peroxide is generated; and
(b) administering to the individual an aldehyde dehydrogenase enzyme that converts acetaldehyde to acetate in a third enzymatic reaction;
(c) allowing the alcohol oxidase, catalase and aldehyde dehydrogenase to react with ethanol and its metabolites in the individual;
so that the concentration of ethanol and its metabolites in the individual is decreased.

2. The method of claim 1, further comprising administering nicotinamide adenine dinucleotide (NAD).

3. The method of claim 2, wherein the aldehyde dehydrogenase and/or the nicotinamide adenine dinucleotide is disposed within a polymeric network configured to form a shell that encapsulates the aldehyde dehydrogenase and/or the nicotinamide adenine dinucleotide.

4. The method of claim 1, wherein the multiple-enzyme nanocomplex system is administered orally.

5. The method of claim 1, wherein the individual suffers from acute ethanol intoxication.

6. The method of claim 1, wherein the multiple-enzyme nanocomplex system is administered parenterally.

7. The method of claim 1, wherein the multiple-enzyme nanocomplex system reduces blood ethanol concentrations in the individual by at least 25, 50, 75 or 100 mg/dL within 90 minutes following administration to the individual.

8. The method of claim 1, wherein the alcohol oxidase enzyme, the catalase enzyme and/or the aldehyde dehydrogenase enzyme is coupled to a polymeric shell or an enzyme within a polymeric shell.

9. The method of claim 1, wherein the polymeric network encapsulates the alcohol oxidase and the catalase in a manner that inhibits degradation of the alcohol oxidase and the catalase when the multiple-enzyme nanocomplex is disposed in an in vivo environment.

10. A composition of matter comprising a multiple-enzyme nanocomplex system for use in a patient for the treatment of a condition resulting from the consumption of alcohol, wherein the multiple-enzyme nanocomplex system comprises:
an alcohol oxidase enzyme that generates hydrogen peroxide and acetaldehyde in a first enzymatic reaction with alcohol;
a catalase enzyme that converts the hydrogen peroxide into water in a second enzymatic reaction;
an aldehyde dehydrogenase enzyme that converts acetaldehyde to acetate in a third enzymatic reaction; and
a polymeric network configured to form a shell that encapsulates the alcohol oxidase and the catalase wherein:
the polymeric network exhibits a permeability sufficient to allow the alcohol to diffuse from an external environment outside of the shell to the alcohol oxidase.

11. The composition of matter of claim 10, wherein the aldehyde dehydrogenase enzyme is disposed within a polymeric network configured to form a shell that encapsulates the aldehyde dehydrogenase.

12. The composition of matter of claim 11, wherein the alcohol oxidase, the catalase and/or the aldehyde dehydrogenase is coupled to a polymeric shell or an enzyme within a polymeric shell.

13. The composition of matter of claim 10, further comprising nicotinamide adenine dinucleotide.

14. The composition of matter system of claim 13, wherein the nicotinamide adenine dinucleotide is disposed within a polymeric network configured to form a shell that encapsulates the nicotinamide adenine dinucleotide

15. A method of making a pharmaceutical composition comprising combining together in an aqueous formulation a multiple-enzyme nanocomplex system comprising:
an alcohol oxidase enzyme that generates hydrogen peroxide and acetaldehyde in a first enzymatic reaction with alcohol;
a catalase enzyme that converts the hydrogen peroxide into water in a second enzymatic reaction:
wherein a polymeric network is disposed around the alcohol oxidase enzyme and the catalase enzyme and configured to form a shell that encapsulates the alcohol oxidase enzyme and the catalase enzyme;
an aldehyde dehydrogenase enzyme that converts acetaldehyde to acetate in a third enzymatic reaction, wherein a polymeric network is disposed around the aldehyde dehydrogenase enzyme and the catalase enzyme and configured to form a shell that encapsulates the aldehyde dehydrogenase enzyme; and
a pharmaceutical excipient selected from the group consisting of:
a preservative, a tonicity adjusting agent, a detergent, a viscosity adjusting agent, a sugar or a pH adjusting agent.

16. The method of claim 15, wherein the polymeric shell of the aldehyde dehydrogenase enzyme comprises moieties capable forming disulfide bonds, and said moieties are reduced.

17. The method of claim 16, wherein the pharmaceutical excipient is selected for use in intravenous administration.

18. The method of claim 17, wherein the aldehyde dehydrogenase enzyme is not disposed within a polymeric network comprising the alcohol oxidase enzyme and the catalase enzyme.

19. The method of claim 18, wherein the multiple-enzyme nanocomplex system further comprises nicotinamide adenine dinucleotide (NAD).

20. The method of claim 19, wherein the zeta potentials of the polymeric shells are selected to be at least ˜1, ˜2 or ˜4 mV at physiological pH.
